(19) United States
(12) Patent Application Publication
(10) Pub. No.: US 2005/0205091 A1
(43) Pub. Date: Sep. 22, 2005

(54) Apparatus for housing a vaporizing substance and method of use therefore

(76) Inventor: Angel Lynn Wygant, Wylie, TX (US)

Correspondence Address: James S. Finn, 1718 M. Street NW, #294, Washington, DC, 20036, US

(21) Appl. No.: 10/802,995

(22) Filed: Mar. 17, 2004

(51) Int. Cl. A61M 15/00; A61M 16/00

(52) U.S. Cl. 128/204.13

(57) ABSTRACT

An apparatus for housing a vaporizing substance, comprising a fabric layer, said fabric layer having a portion thereon for placement of a vaporizing substance, a cover associated with said fabric layer capable of being placed over said area for said vaporizing substance, said cover formed of gas permeable material, and an attaching device for attaching said fabric layer to a user.

### BACKGROUND OF THE INVENTION

Vaporizing substances are utilized in a number of ways to the benefit of the user. However, in order to utilize these substances they should be placed in close proximity to the user's nose, combined with hot water and the vaporized steam inhaled by the user, rubbed onto the skin of the user or placed on a device which is adhered to the skin or clothing of the user and the vapors inhaled.

Combining the vaporizing substance with hot water and breathing the steam is an effective delivery method but has several disadvantages. First, it is messy since the face of the user must be placed directly over the steam. Second, it requires the user to be near a hot water in order to combine hot water with the vaporizing substance to produce the steamy vapors. Third, the user must be physically located in an appropriate location that allows the user to hover for a period of time over the steaming, vaporized liquid. There are many situations where this procedure is not possible, practical or convenient. For these reasons, this method can be undesirable.

Another method for using vaporizing substances is to rub vaporizing ointment directly onto the skin of the user. The disadvantages of this method are numerous. First, placing the ointment directly on the skin may cause skin irritation, particularly in individuals with sensitive skin. Second, it is rather messy since the ointment is not covered by any other material potentially allowing the ointment to come into contact with and soil clothing and other materials. Third, it is a longer-term solution. In order to remove the vaporization effect, the ointment must be washed off the skin as well as any other material it has come into contact with during the duration of the treatment. In addition, the ointment can inadvertently be touched and transferred to other parts of the body such as the eyes or mouth causing, at a minimum, potential discomfort for the user. Finally, the user must be physically located in an appropriate location that allows the user to wear the ointment for extended periods. There are many situations where this procedure is not possible, practical or convenient. For these reasons, this method can be undesirable.

Therapeutic wraps that apply ointment to the neck and chest of a user effectively serve to protect clothing and other materials from coming in contact with the ointment but still require applying the ointment to the skin of the user. This presents a problem for users who have sensitive skin. In addition, this solution is also a longer-term treatment solution since the ointment comes in contact with the skin and cannot be readily removed without washing the ointment off of the skin. For these reasons, this method can be undesirable.

“Patches”, “Strips”, or other adhesive devices containing substances that produce vapors can be undesirable to those who have skin sensitivities or do not wish to have adhesive material come into contact with their skin or clothing. In addition, the vaporizing substance is exposed and can be touched and transferred to other areas of the body. For this reason, this method can be undesirable.

### SUMMARY OF THE INVENTION

In one preferred embodiment, the present invention provides an apparatus for securing a substance proximately to a predefined area on a user's body, comprising a layer of material capable of being attached to said user, a substance placement area located on said layer of material, and a cover associated with said layer of material capable of being placed over said substance placement area, said cover formed of gas permeable material. In a preferred embodiment of the present invention, the predetermined area on said user's body is the user's nasal passage and the layer of material is a layer of fabric shaped as a baby bib. The layer of material capable of being attached to said user may be attached to said user by a hook and loop fastener, safety pins, zipper assembly, snaps, stitching or ties and the substance placement area located on said layer of material is a vaporizing substance. Further, the placement area located on said layer of material can include a slot integral to said layer of material into which is placed a device containing a vaporizing substance.

In yet another preferred embodiment of the present invention is provided a method of housing a vaporizing substance in proximity to a user's nasal passage, comprising providing a fabric layer, said fabric layer having a portion thereon for placement of a vaporizing substance, covering said vaporizing substance with a gas permeable material, and attaching said fabric layer to a user. The fabric layer of a preferred embodiment of the present method may be bib shaped and the step of attaching said fabric layer to user may include placing an opening in said fabric layer around the neck of the user and said step of attaching said fabric layer to a user can include, in a preferred embodiment of the present invention placing an opening in said fabric layer around the neck of the user wherein said opening in said fabric for placement around the neck of the user is formed by joining two mating longitudinal fabric members integral to and at one end of said fabric and attached together around a users neck by a hood and loop fastener. The area for receiving said vaporizing substance can include a leak-resistant material that prevents the vaporizing substance from coming in contact with the body of the user. Also, the portion of said fabric layer for placement of a vaporizing substance may be, in one preferred embodiment, a slot integral to said fabric layer into which is placed a device containing said vaporizing substance. The attaching device may be a hook and loop fastener, safety pins, zipper assembly, snaps, stitching or ties.

In yet another preferred embodiment of the present invention is provided an apparatus for housing a vaporizing substance, comprising a fabric layer, said fabric layer having a portion thereon for placement of a vaporizing substance, a cover associated with said fabric layer capable of being placed over said area for said vaporizing substance, said cover formed of gas permeable material, and an attaching device for attaching said fabric layer to a user.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 illustrates a top view of a preferred embodiment of the present invention; and

FIG. 2 depicts a side elevation of a preferred embodiment of the present invention wherein the gas permeable material is unfastened and lifted exposing the area for receiving the vaporizing substance.

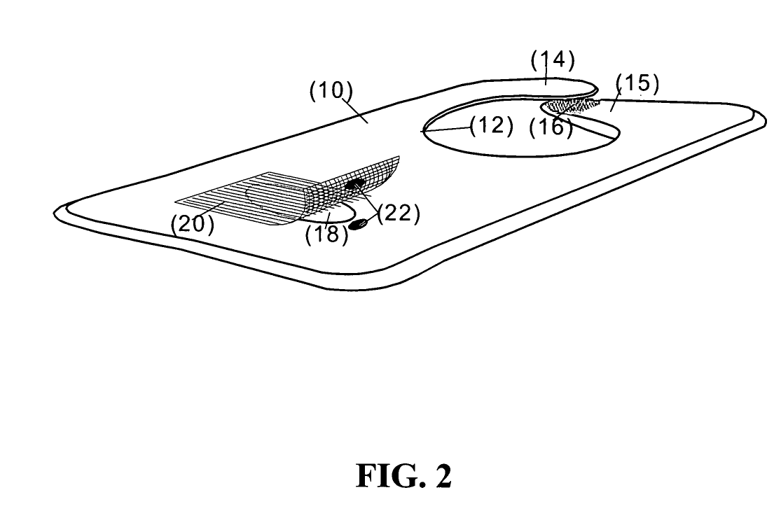

It will be appreciated that for simplicity and clarity of illustration, elements illustrated in the figures have not necessarily been drawn to scale. For example, the dimensions of some of the elements are exaggerated relative to other elements for clarity. Further, where considered appropriate, reference numerals have been repeated among the figures to indicate corresponding or analogous elements.

### DESCRIPTION OF THE PREFERRED EMBODIMENT

In the following detailed description, numerous specific details are set forth in order to provide a thorough understanding of the invention. However, it will be understood by those skilled in the art that the present invention may be practiced without these specific details.

It will be appreciated that one embodiment of the present invention keeps a given substance, such as a vaporizing substance from being applied directly to the skin by allowing the substance to be applied to an area of the apparel. The vapor bib device can be worn for very short periods as well as extended periods at the discretion of the user and, thus, the benefits of the vapor treatment can then be gained at the convenience of the user, then quickly and easily removed with no mess.

Also, in one preferred embodiment of the present invention the vapor bib device containing the vaporizing substance may be covered by a gas permeable material preventing the user from inadvertently touching the vaporizing and transferring it to other areas of the body.

The vapor bib device allows the user to easily and conveniently access the therapeutic benefits of breathing the vapors emitted by the vaporizing substance in virtually any location, for varying periods of time and without allowing the vaporizing substance or adhesives associated with other devices to come into contact with the skin of the user and preventing inadvertent transfer of the vaporizing substance to other areas of the body of the user.

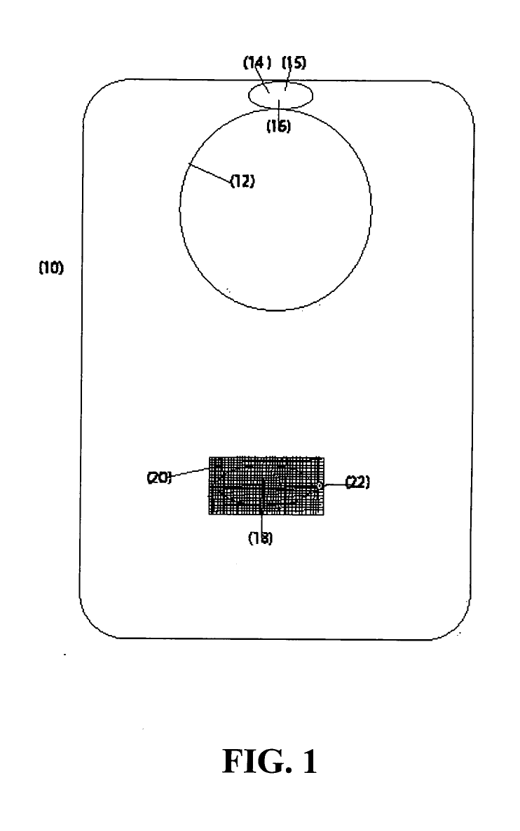

Turning now to the figures, a top view of a preferred embodiment of the present invention is illustrated in FIG. 1. The vapor bib device is made of a given material, such as fabric 10 and in a preferred embodiment may be least 4 inches wide with the vertical length of the face of the material is at least 4 inches. It contains an opening the diameter of which is at least 4 inches from the user's neck 12 with 2 mating pieces 14 and 15 that fasten 16 behind the neck of the user with a fastening device that holds the bib in place but is relatively simple to unfasten. Although the given material in the above preferred embodiment is fabric, it is understood that a cloth-like material can also be used. Further, this cloth like material can be disposable or non-disposable.

The area for receiving the vaporizing substance 18 is attached to the body of the bib and is made of moisture-resistant and leak-resistant material that may be about ½″ in diameter. The area for receiving the vaporizing substance is then covered 20 with a gas permeable material which is attached to the body of the bib 10 and is fastened 22 to the body of the bib 10 on the other side of the area for receiving the vaporizing substance 18 in such a way that it is secured in place but is relatively simple to unfasten. Also, the portion of said fabric layer for placement of a vaporizing substance may be, in another preferred embodiment, a slot integral to the fabric layer into which is placed a device containing the vaporizing substance.

The fastener 22 can be a hook and loop fastener (such as Velcro®), safety pins, zipper assembly, snaps, ties, stitching or any other fastening means known to those of ordinary skill in the fastening art. Also, in another preferred embodiment of the present invention, there may be no need for a fastener as a hole, large enough to be placed over the head of a user may be contained in the material. Thus, for use the user simply slips the device over their head.

The manner of using the vapor bib is to place vaporizing substance into the area designated for receiving the vaporizing substance, securing the gas permeable material over said area and fastening it closed. The preferred embodiment of the bib is to place it around the neck of the user and fasten the mating pieces behind the neck. This operation places the vapors in an appropriate manner to optimize the ability of the user to breath in the vapors without having the vaporizing substance rubbed upon the skin of the user or inadvertently touched and transferred to other parts of the body as well as materials in close proximity to the user. When the user has completed the treatment session, the bib can be removed by unfastening the neck fastener. The user may then proceed into environments where the vapors emitted by the vaporizing substance might not be desirable or allowed. The bib can be reused in its current state for as long as the vaporizing substance remains on the in the area for receiving of vaporizing substance. When the user is finished with the vapor bib device for treatment purposes, it can be washed and stored in the same manner as any other bib.

Accordingly, the reader will see that the present invention described above allows the user to derive the therapeutic benefits of breathing vapors to treat nasal congestion while avoiding the mess, skin irritation and potential misplacement of the vapors by preventing the substance from inadvertently being transferred to other areas of the body (such as the eyes or mouth) or soiling materials which are in close proximity to the user. Furthermore, the vapor bib has the following additional advantages:
it is cost-effective in that the same device can be cleaned and re-used for future applications;
allows the user to conserve vaporizing substance by potentially using and re-using a smaller amount for multiple treatments;
allows the user to participate in short or long treatment sessions held at intermittent intervals over an extended period of time by removing and replacing the vapor bib device at the discretion of the user;
it can be manufactured in a variety of colors, designs and shapes making it's use more acceptable in a greater number of environments;

it frees the hand of the user during the treatment allowing the user to engage in other tasks during the treatment
the vapor bib device can serve as a protective device without vaporizing substance when treatments are not necessary

While various embodiments of the present invention have been described above, it should be understood that they have been presented by way of example, and not limitation. It will be apparent to persons skilled in the relevant art that various changes in form and detail can be made therein without departing from the spirit and scope of the invention. For example, the vapor bib device can have other shapes, colors, apparel types such as vests, shirts, etc. Thus the scope of the invention should be determined by the appended claims and their legal equivalents, rather than by the examples given.

1. An apparatus for housing a vaporizing substance, comprising:
a fabric layer, said fabric layer having a portion thereon for placement of a vaporizing substance;
a cover associated with said fabric layer capable of being placed over said area for said vaporizing substance, said cover formed of gas permeable material; and
an attaching device for attaching said fabric layer to a user.

2. The apparatus for housing a vaporizing substance of claim 1, wherein said fabric layer is bib shaped.

3. The apparatus for housing a vaporizing substance of claim 1, wherein said attaching device for attaching said fabric layer to said user is an opening in said fabric for placement around the neck of the user.

4. The apparatus for housing a vaporizing substance of claim 3, wherein said attaching device for attaching said fabric layer to said user is an opening in said fabric for placement around the neck of said user, said opening in said fabric is formed by joining two mating longitudinal fabric members integral to and at one end of said fabric and attached together around a users neck by a hood and loop fastener.

5. The apparatus for housing a vaporizing substance of claim 3, wherein said area for receiving said vaporizing substance comprises leak-resistant material that prevents the vaporizing substance from coming in contact with the body of the user.

6. The apparatus for housing a vaporizing substance of claim 1, wherein said portion of said fabric layer for placement of a vaporizing substance consists of a slot integral to said fabric layer into which is placed a device containing said vaporizing substance.

7. The apparatus for housing a vaporizing substance of claim 1, wherein said attaching device is a hook and loop fastener, safety pins, zipper assembly, snaps, stitching or ties.

8. A method of housing a vaporizing substance in proximity to a user's nasal passage, comprising:
providing a fabric layer, said fabric layer having a portion thereon for placement of a vaporizing substance;
covering said vaporizing substance with a gas permeable material; and
attaching said fabric layer to a user.

9. The method of housing a vaporizing substance of claim 1, wherein said fabric layer is bib shaped.

10. The method of housing a vaporizing substance of claim 1, wherein the step of attaching said fabric layer to a user includes placing an opening in said fabric layer around the neck of the user.

11. The method of housing a vaporizing substance of claim 10, wherein said step of attaching said fabric layer to a user includes placing an opening in said fabric layer around the neck of the user wherein said opening in said fabric for placement around the neck of the user is formed by joining two mating longitudinal fabric members integral to and at one end of said fabric and attached together around a users neck by a hood and loop fastener.

12. The method of housing a vaporizing substance of claim 10, wherein said area for receiving said vaporizing substance comprises leak-resistant material that prevents the vaporizing substance from coming in contact with the body of the user.

13. The method of housing a vaporizing substance of claim 10, wherein said portion of said fabric layer for placement of a vaporizing substance consists of a slot integral to said fabric layer into which is placed a device containing said vaporizing substance.

14. The method of housing a vaporizing substance of claim 10, wherein said attaching device is a hook and loop fastener, safety pins, zipper assembly, snaps, stitching or ties.

15. An apparatus for securing a substance proximately to a predefined area on a user's body, comprising:
a layer of material capable of being attached to said user;
a substance placement area located on said layer of material; and
a cover associated with said layer of material capable of being placed over said substance placement area, said cover formed of gas permeable material.

16. The apparatus for securing a substance proximately to a predefined area on a user's body of claim 15, wherein said predetermined area on said user's body is the user's nasal passage.

17. The apparatus for securing a substance proximately to a predefined area on a user's body of claim 15, wherein said layer of material is a layer of fabric shaped as a baby bib.

18. The apparatus for securing a substance proximately to a predefined area on a user's body of claim 15, wherein said layer of material capable of being attached to said user is attached to said user by a hook and loop fastener, safety pins, zipper assembly, snaps, stitching or ties.

19. The apparatus for securing a substance proximately to a predefined area on a user's body of claim 15, wherein on said substance placement area located on said layer of material is placed a vaporizing substance.

20. The apparatus for securing a substance proximately to a predefined area on a user's body of claim 15, wherein said substance placement area located on said layer of material includes a slot integral to said layer of material into which is placed a device containing a vaporizing substance.

21. The apparatus for housing a vaporizing substance of claim 1, wherein said fabric layer is constructed of a disposable cloth-like material.

22. The apparatus for housing a vaporizing substance of claim 21, wherein said disposable cloth-like material is bib-shaped.
